(19) United States
(12) Patent Application Publication
(10) Pub. No.: US 2026/0229348 A1
(43) Pub. Date: Aug. 6, 2026

(54) YOUTHFULNESS DEGREE OUTPUT DEVICE AND YOUTHFULNESS DEGREE OUTPUT METHOD

(71) Applicant: NTT DOCOMO, INC., Tokyo (JP)

(72) Inventors: Takashi HAMATANI, Chiyoda-ku (JP); Miyari HATAMOTO, Chiyoda-ku (JP)

(73) Assignee: NTT DOCOMO, INC., Tokyo (JP)

(21) Appl. No.: 19/143,480

(22) PCT Filed: Nov. 8, 2023
(86) PCT No.: PCT/JP2023/040291

§ 371 Date: Jun. 26, 2025

(30) Foreign Application Priority Data
Jan. 13, 2023 (JP) 2023-003581

(51) Int. Cl. G16H 40/63

(52) U.S. Cl. CPC G16H 40/63

(57) ABSTRACT

The object is to output the youthfulness degree of a user. A youthfulness degree output device 1 includes: a storage unit 10 that stores a scoring model for predicting the youthfulness degree of a user by inputting the user's age and a log frequency related to logs associated with the user's behavior obtained by a mobile terminal 2 carried by the user; an acquisition unit 11 that acquires a target user's age and the log frequency; and an output unit 13 that outputs the youthfulness degree of the target user predicted by inputting the target user's age and the log frequency acquired by the acquisition unit 11 to the scoring model stored in the storage unit 10.

### TECHNICAL FIELD

One aspect of the present disclosure relates to a youthfulness degree output device and a youthfulness degree output method for outputting the youthfulness degree of a user.

### BACKGROUND ART

The following Patent Literature 1 discloses an information processing device that calculates the biological age of each of a plurality of samples (subjects) by applying a biological age prediction model to the evaluation value of each sample with reference to a database in which the actual age, the evaluation value of each item of biometric information reflecting lifestyle habits, and the actual value of each item of lifestyle habits are associated with each other.

### CITATION LIST

### Patent Literature

Patent Literature 1: Japanese Unexamined Patent Publication No. 2022-142021

### SUMMARY OF INVENTION

### Technical Problem

Although the information processing device calculates the biological age of the sample, it is not possible to output the youthfulness degree of the sample. Therefore, it is desirable to output the youthfulness degree of the user.

### Solution to Problem

A youthfulness degree output device according to an aspect of the present disclosure includes: a storage unit that stores a prediction model for predicting a youthfulness degree of a user by inputting the user's age and a log frequency related to logs associated with the user's behavior obtained by a terminal carried by the user; an acquisition unit that acquires a target user's age and the log frequency; and an output unit that outputs a youthfulness degree of the target user predicted by inputting the target user's age and the log frequency acquired by the acquisition unit to the prediction model stored in the storage unit. The prediction model predicts a youthfulness degree based on a cumulative distribution function for each age calculated by counting the log frequency for each of a plurality of users by age.

According to such an aspect, the youthfulness degree of the target user predicted by inputting the target user's age and the log frequency to the prediction model is output. That is, it is possible to output the youthfulness degree of the user.

### Advantageous Effects of Invention

According to one aspect of the present disclosure, it is desirable to output the youthfulness degree of the user.

### BRIEF DESCRIPTION OF DRAWINGS

FIG. 1 A diagram showing an example of the system configuration of a youthfulness degree output system including a youthfulness degree output device according to an embodiment.

FIG. 2 A diagram showing an example of a log table.

FIG. 3 A diagram showing an example of the functional configuration of the youthfulness degree output device according to the embodiment.

FIG. 4 A diagram showing an example of a graph of the frequency of the number of screen-on times.

FIG. 5 A diagram showing an example of the probability density distribution of the number of screen-on times.

FIG. 6 A diagram showing an example of the cumulative distribution of the number of screen-on times.

FIG. 7 A diagram showing an example of a graph of the frequency of the average unlock time.

FIG. 8 A diagram showing an example of the probability density distribution of the average unlock time.

FIG. 9 A diagram showing an example of the cumulative distribution of the average unlock time.

FIG. 10 A diagram showing an example of a graph of the frequency of the number of steps.

FIG. 11 A diagram showing an example of the probability density distribution of the number of steps.

FIG. 12 A diagram showing an example of the cumulative distribution of the number of steps.

FIG. 13 A diagram showing an example of a graph of the frequency of the area of the living scope.

FIG. 14 A diagram showing an example of the probability density distribution of the area of the living scope.

FIG. 15 A diagram showing an example of the cumulative distribution of the area of the living scope.

FIG. 16 A diagram showing an example of a graph of a median of the number of screen-on times by age.

FIG. 17 A diagram showing an example of interpretation of the graph shown in FIG. 16.

FIG. 18 A diagram showing an example of interpretation of the cumulative distribution of the number of screen-on times.

FIG. 19 A diagram showing an example of a graph of a median of the average screen-on time by age.

FIG. 20 A diagram showing an example of interpretation of the graph shown in FIG. 19.

FIG. 21 A diagram showing an example of interpretation of the cumulative distribution of the average screen-on time.

FIG. 22 A diagram showing an example of the flow of a scoring model creation process.

FIG. 23 A flowchart showing an example of the scoring model creation process performed by the youthfulness degree output device according to the embodiment.

FIG. 24 A diagram showing an example of the flow of a score calculation process.

FIG. 25 A diagram showing another example of the flow of the score calculation process.

FIG. 26 A flowchart showing an example of the score calculation process performed by the youthfulness degree output device according to the embodiment.

FIG. 27 A flowchart showing an example of a youthfulness degree output process performed by the youthfulness degree output device according to the embodiment.

FIG. 28 A diagram showing an example of the hardware configuration of a computer used in the youthfulness degree output device according to the embodiment.

### DESCRIPTION OF EMBODIMENTS

Hereinafter, embodiments of the present disclosure will be described in detail with reference to the diagrams. In addition, in the description of the diagrams, the same elements are denoted by the same reference numerals, and repeated description thereof will be omitted. In addition, the embodiments of the present disclosure in the following description are specific examples of the present invention, and the present invention is not limited to these embodiments unless there is a statement that specifically limits the present invention.

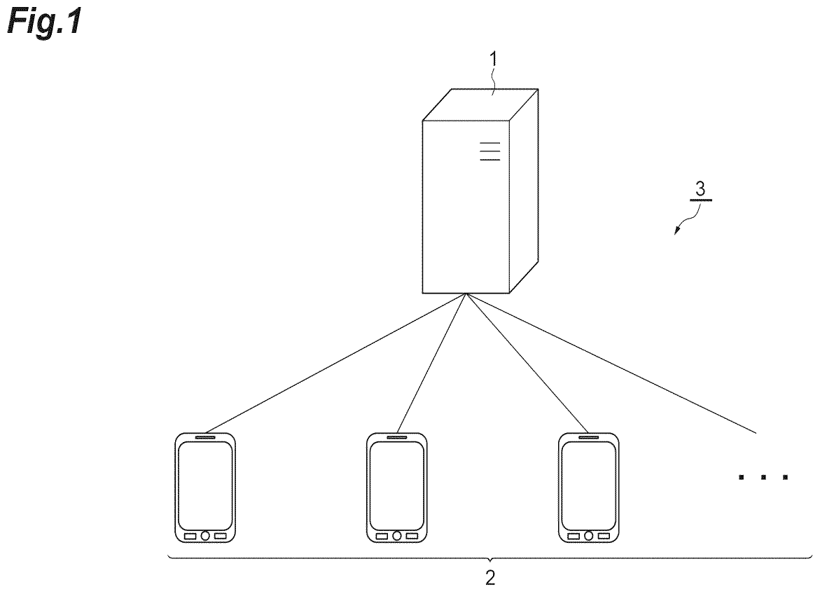

FIG. 1 is a diagram showing an example of the system configuration of a youthfulness degree output system 3 including a youthfulness degree output device 1 according to an embodiment. As shown in FIG. 1, the youthfulness degree output system 3 includes the youthfulness degree output device 1 and one or more mobile terminals 2. In the embodiment, one or more mobile terminals 2 will be collectively referred to as a “mobile terminal 2” as appropriate. The youthfulness degree output device 1 and each mobile terminal 2 are communicatively connected to each other through a network, such as a mobile communication network, so that information can be transmitted and received to and from each other.

The youthfulness degree output device 1 is a computer device that outputs the youthfulness degree of a target user.

The target user is a user as a target. More specifically, the target user is a user (person) of the youthfulness degree output device 1 or a person who indirectly uses the youthfulness degree output device 1, who is the target of the youthfulness degree to be output.

The youthfulness degree is the degree (level, dimension, stage, score) of youthfulness. Youthfulness, for example, does not have to be based on actual age. The youthfulness degree may be, for example, a value indicating how young a behavior performed by the user is (whether it can be said that the behavior is more likely to be performed by younger people). The youthfulness degree may be, for example, a value indicating the degree to which a behavior performed by the user deviates from the normally expected behavior or a value indicating the good or bad condition of the behavior. The youthfulness degree may be, for example, a real number of “0” or more and “1” or less, with the closer to “O” being less young (older) and the closer to “1” being younger. The youthfulness degree may be, for example, a probability of “0%” or more and “100%” or less, with the closer to “0%” being less young (older) and the closer to “100%” being younger. The youthfulness degree may also be a value for evaluating the cognitive function of the user. The youthfulness degree may be a value indicating brain age, which is an indicator of brain function.

The details of the youthfulness degree output device 1 will be described later.

The mobile terminal 2 is a mobile communication terminal for performing mobile communication or a computer device such as a notebook computer. In the embodiment, the mobile terminal 2 is assumed to be a smartphone, but is not limited thereto. The mobile terminal 2 is carried by the user.

The mobile terminal 2 may include a GPS (global positioning system), and may acquire current location information (latitude, longitude, and the like) of the mobile terminal 2 using the GPS. In addition, the mobile terminal 2 may acquire current location information based on base station information without using the GPS.

The mobile terminal 2 may include various sensors, and may collect log frequencies (described later) related to (automatically obtainable) logs associated with the behaviors (operations, movements, actions) of the user carrying the mobile terminal 2 by using various sensors. The mobile terminal 2 may collect the log frequency by using the function of the installed OS (Operating System). An arbitrary app (application) may be installed on the mobile terminal 2, and the log frequency may be collected by using the app. The mobile terminal 2 may include other sensors and functions that are included in a typical smartphone, and may collect various log frequencies by using these sensors and functions.

The log may be a log related to the user's operation on the mobile terminal 2. For example, the log may be a log related to screen on, screen off, screen on/off, screen unlock, app launch, app exit, app installation, or app use. In this case, the log frequency may be the number of screen-on times, the average screen-on time, the number of screen-off times, the average screen-off time, the number of screen on/off times, the number of screen unlock times, the average screen unlock time, the average time required to unlock the screen, the number of times of app launch, the number of times of app exit, the number of app categories (based on the user's operation to install the app), the average app use time, or the average time from unlocking the screen to launching an app.

The log frequency may be a frequency within a period (for example, one day) that is set in advance in the youthfulness degree output system 3. For example, the number of screen-on times may be the number of times the user turns on the screen of the mobile terminal 2 in one day (for example, in units of times/day). The log frequency may be a value converted into a frequency. For example, when the number of screen-on times is “1” or more and “5” or less, this be defined as a frequency “1”, and when the number of screen-on times is “6” or more and “10” or less, this be defined as a frequency “2”. Conversion into a frequency may be appropriately performed in the mobile terminal 2, or may be appropriately performed (by each functional block) in the youthfulness degree output device 1. In the embodiment, the “log frequency” may be appropriately regarded as a value converted into a frequency, or may be appropriately regarded as a value that is not converted into a frequency.

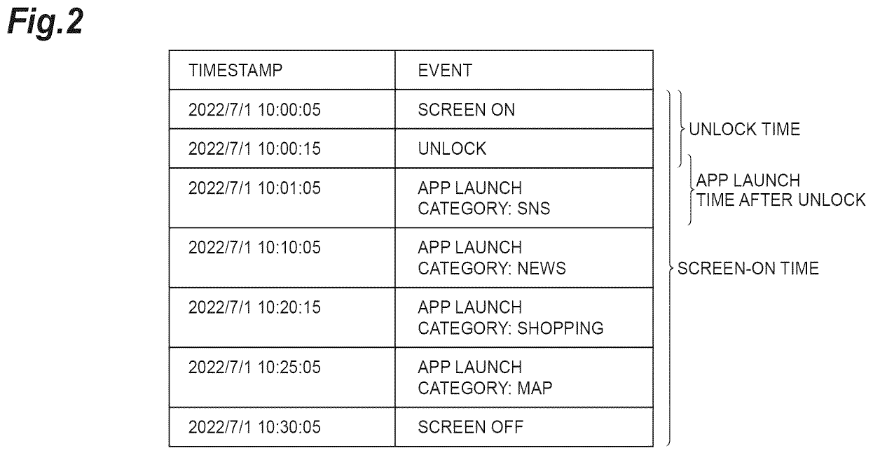

FIG. 2 is a diagram showing a table example of a log. In the table example shown in FIG. 2, a timestamp, which is a date and time, is associated with an event, which is information indicating an operation performed by the user at that date and time. The log shown in FIG. 2 may be stored (accumulated) in each mobile terminal 2. The mobile terminal 2 may calculate a log frequency based on the log. For example, as shown in FIG. 2, the time from the event “screen on” to the next event “screen off” is calculated as a screen-on time. In addition, for example, the time from the event “screen on” to the next event “(screen) unlock” is calculated as a (screen) unlock time. In addition, for example, the time from the event “(screen) unlock” to the next event “app launch” may be calculated as an app launch time after (screen) unlock.

The log may be a log related to the user's own operation. For example, the log may be a log regarding location information according to the movement of the user. In this case, the log frequency (which is also a feature obtained from location information) may be the number of steps, the area of the living scope, the number of times of going out, the average (moving) distance from home, the number of stay points, the tendency of the number of people at the stay points, the number of areas visited, and the sum of the number of other visitors in each area visited.

The mobile terminal 2 outputs (transmits) the user's age, which is stored (registered, set) in advance in the mobile terminal 2, and the collected log frequency to the youthfulness degree output device 1 through the network. The log frequency to be output may be a plurality of types instead of one type. For example, the mobile terminal 2 may output the user's age, the number of screen-on times, the average screen unlock time, the number of steps, and the area of the living scope (a total of four types of log frequencies) to the youthfulness degree output device 1. The output timing may be periodic (for example, once a day), or may be any timing designated by the youthfulness degree output device 1 or the mobile terminal 2. When outputting the age and the log frequency to the youthfulness degree output device 1, the mobile terminal 2 may output the age and the log frequency together with other arbitrary information. The mobile terminal 2 may output the user's age and the log (that is not the log frequency) to the youthfulness degree output device 1.

In addition, in the embodiment, “age” may be appropriately replaced with “frequency of age”. The frequency of age is a value converted into a frequency. For example, the 60s (60 to 69 years old) are defined as “6” and the 70s (70 to 79 years old) are defined as “7”. In addition, the age may be appropriately converted into the frequency of age in the mobile terminal 2, or the age may be appropriately converted into the frequency of age in the youthfulness degree output device 1. In the embodiment, “age” may be appropriately regarded as a value converted into a frequency, or may be appropriately regarded as a value that is not converted into a frequency.

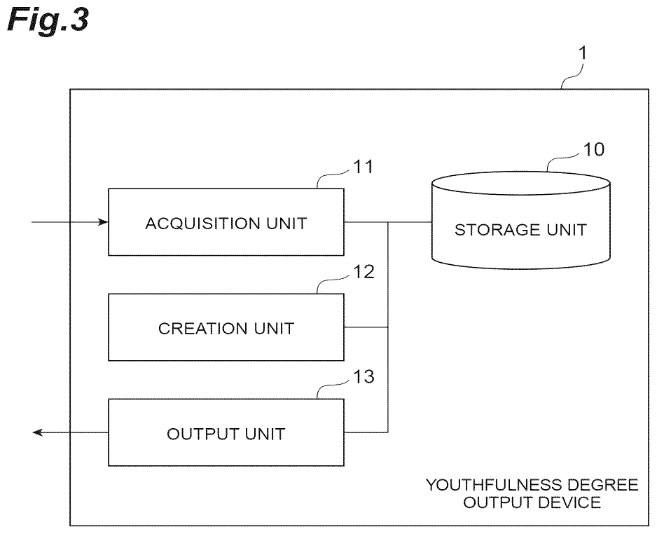

FIG. 3 is a diagram showing an example of the functional configuration of the youthfulness degree output device 1. As shown in FIG. 3, the youthfulness degree output device 1 includes a storage unit 10 (storage unit), an acquisition unit 11 (acquisition unit), a creation unit 12 (creation unit), and an output unit 13 (output unit).

Each functional block of the youthfulness degree output device 1 is assumed to function in the youthfulness degree output device 1, but is not limited thereto. For example, some of the functional blocks of the youthfulness degree output device 1 may function within a computer device, which is different from the youthfulness degree output device 1 and connected to the youthfulness degree output device 1 through the network, while appropriately transmitting and receiving information to and from the youthfulness degree output device 1. In addition, some of the functional blocks of the youthfulness degree output device 1 may be omitted, a plurality of functional blocks may be integrated into one functional block, or one functional block may be separated into a plurality of functional blocks.

Hereinafter, each function of the youthfulness degree output device 1 shown in FIG. 3 will be described.

The storage unit 10 stores arbitrary information used for calculations in the youthfulness degree output device 1, the results of calculations in the youthfulness degree output device 1, and the like. The information stored in the storage unit 10 may be referred to as appropriate by each function of the youthfulness degree output device 1.

The storage unit 10 stores a scoring model (prediction model) (described later) that predicts the youthfulness degree of a user by receiving, as its input, the user's age and a log frequency related to logs associated with the user's behavior obtained by the mobile terminal 2 carried by the user. The storage unit 10 may store a scoring model created by the creation unit 12 (described later). The storage unit 10 may store a scoring model corresponding to each of a plurality of types of log frequencies.

The acquisition unit 11 acquires the target user's age and the log frequency (of the target user). The target user may be set in advance in the youthfulness degree output system 3, or may be designated by the user (including the target user himself or herself) or an administrator of the youthfulness degree output device 1. The acquisition unit 11 may acquire the target user's age and a plurality of types of log frequencies (of the target user). The acquisition unit 11 may acquire the target user's age and (one or more types of) log frequency from the mobile terminal 2 of the target user through a network, or may acquire the same from the storage unit 10 (where these are stored in advance). The acquisition unit 11 may cause the storage unit 10 to store the acquired target user's age and (one or more types of) log frequency, or may output the same to another functional block.

The acquisition unit 11 may acquire the age of any or all users and (one or more types of) log frequency (of the users) (to create or update the scoring model). The acquisition unit 11 may cause the storage unit 10 to store the acquired target user's age and (one or more types of) log frequency, or may output the same to another functional block.

The acquisition unit 11 may acquire a log instead of a log frequency. In this case, the youthfulness degree output device 1 (each functional block) may calculate a log frequency based on the acquired log, and the calculated log frequency may be used in the processing of the youthfulness degree output device 1.

The creation unit 12 creates a scoring model. The creation unit 12 may cause the storage unit 10 to store the created scoring model, or may output the same to another functional block.

As described above, the scoring model predicts the youthfulness degree of the user by inputting the user's age and a log frequency related to logs associated with the user's behavior obtained by the mobile terminal 2 carried by the user. The scoring model may predict the youthfulness degree based on a cumulative distribution function for each age that is calculated by counting the log frequencies of a plurality of users by age. The scoring model may predict the youthfulness degree by applying the input log frequency to the cumulative distribution function of the input age among the cumulative distribution functions by age.

The cumulative distribution function may be calculated (by the creation unit 12) based on the correlation between age and log frequency. The cumulative distribution function may be calculated by accumulating the probability for each log frequency from the lower limit to the upper limit of the log frequency (by the creation unit 12) when there is a negative correlation between age and log frequency, and may be calculated by accumulating the probability for each log frequency from the upper limit to the lower limit of the log frequency (by the creation unit 12) when there is a positive correlation between age and log frequency. The scoring model may predict a value based on a probability that the log frequency is equal to or less than the input log frequency, as the youthfulness degree, when there is a negative correlation between age and log frequency, and may predict a value based on a probability that the log frequency is equal to or greater than the input log frequency, as the youthfulness degree, when there is a positive correlation between age and log frequency.

Hereinafter, the details of the scoring model and the method of creation by the creation unit 12 will be specifically described.

First, a probability density distribution (probability density distribution function) and a cumulative distribution (cumulative distribution function) will be described.

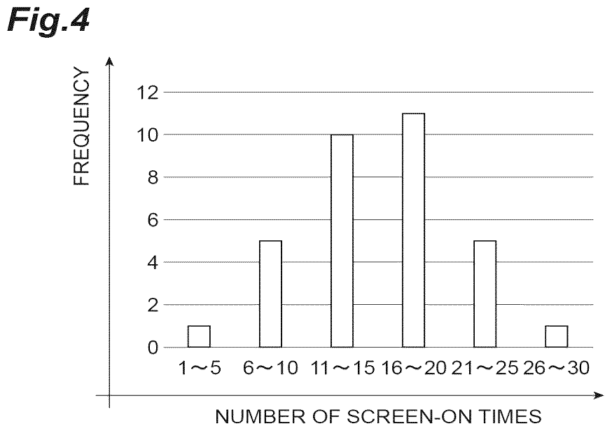

FIG. 4 is a diagram showing an example of a graph of the frequency of the number of screen-on times. The graph shown in FIG. 4 is a graph calculated by counting the number of screen-on times (log frequency) of each user of a predetermined age (for example, 20 years old) among a plurality of users. The horizontal axis of the graph indicates the number of screen-on times. The vertical axis of the graph indicates frequency, which is the number of data pieces present within the range of the number of screen-on times (the number of users corresponding to the number of screen-on times). For example, the graph shown in FIG. 4 indicates that the frequency of the number of screen-on times of a user of a predetermined age (for example, 20 years old) whose number of screen-on times in a period (for example, one day) set in advance is “11” to “15” is “10”.

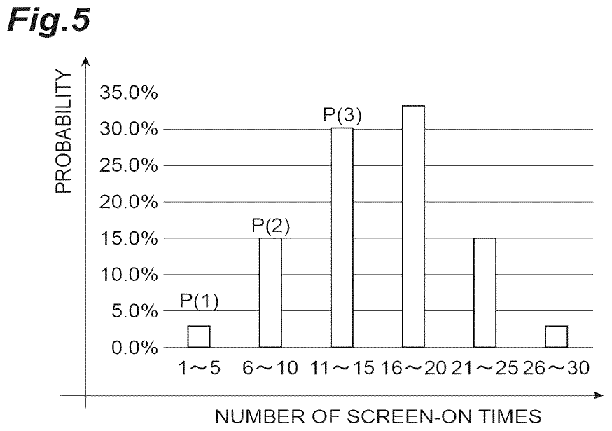

FIG. 5 is a diagram showing an example of the probability density distribution of the number of screen-on times. The probability density distribution shown in FIG. 5 is a probability version of the graph shown in FIG. 4. The probability density distribution indicates a probability distribution for each log frequency (frequency). In the probability density distribution, the sum of each log frequency (frequency) is “1”. The creation unit 12 creates a probability density distribution for each attribute such as age. The probability density distribution is expressed as P(X) (X is a log frequency).

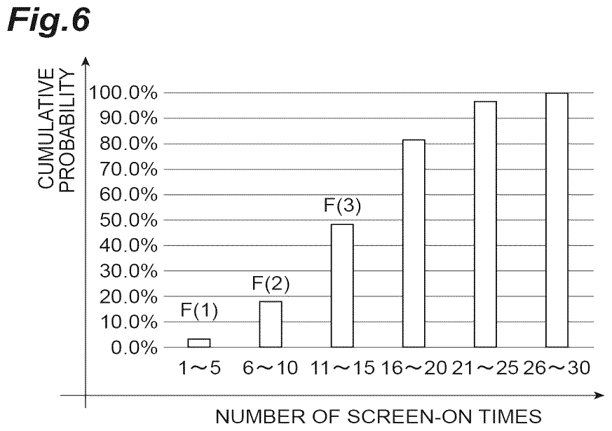

FIG. 6 is a diagram showing an example of the cumulative distribution of the number of screen-on times. The cumulative distribution shown in FIG. 6 is obtained by stacking the probability density distributions shown in FIG. 5. The cumulative distribution is a one-way accumulation of the probability for each log frequency (frequency). The cumulative distribution is generally a function that expresses a probability that a random variable will be equal to or less than a predetermined value. The creation unit 12 creates a cumulative distribution for each attribute such as age. The cumulative distribution is expressed by the following equation.

$$F ( x ) - {\sum }_{X \leq x} P ( X ) \qquad [ Equation 1 ]$$

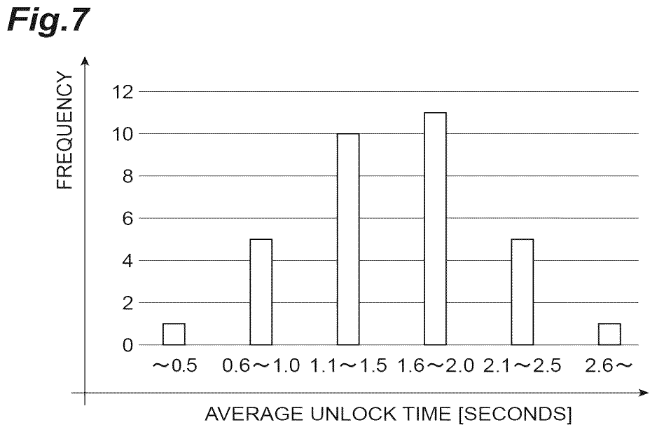

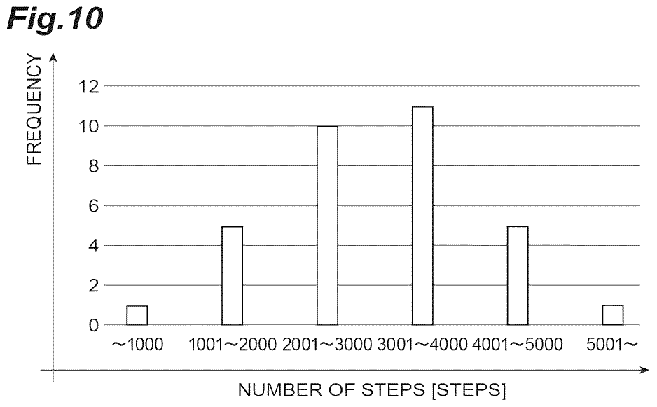

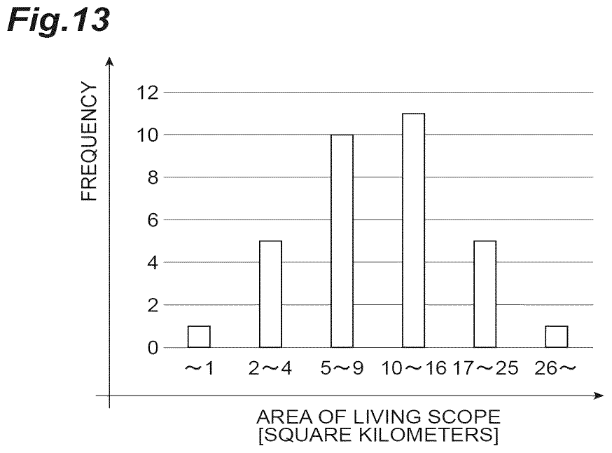

FIGS. 4 to 6 are diagrams related to the number of screen-on times, while FIGS. 7 to 9 are similar diagrams related to the average unlock time, FIGS. 10 to 12 are similar diagrams related to the number of steps, and FIGS. 13 to 15 are similar diagrams related to the area of the living scope. Since these are similar diagrams, detailed explanations thereof will be omitted below.

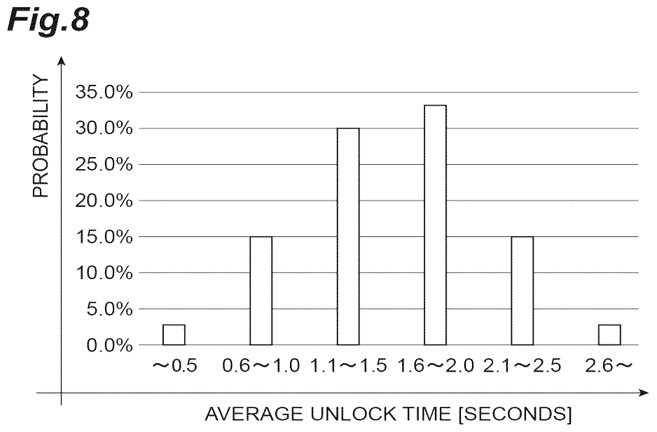

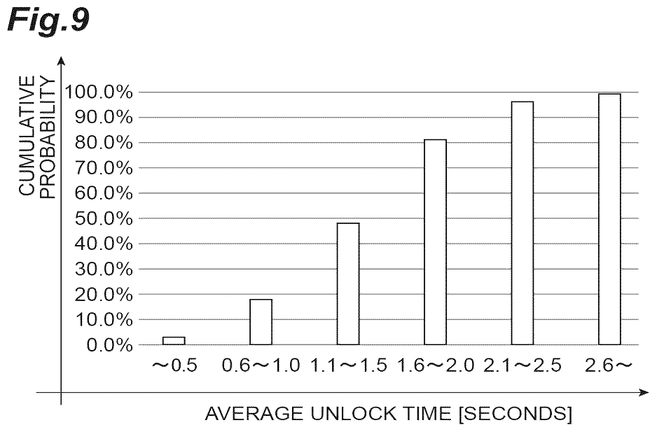

FIG. 7 is a diagram showing an example of a graph of the frequency of the average unlock time. FIG. 8 is a diagram showing an example of the probability density distribution of the average unlock time. FIG. 9 is a diagram showing an example of the cumulative distribution of the average unlock time.

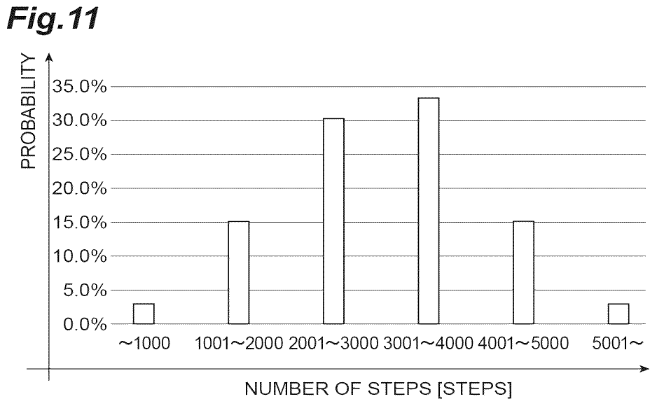

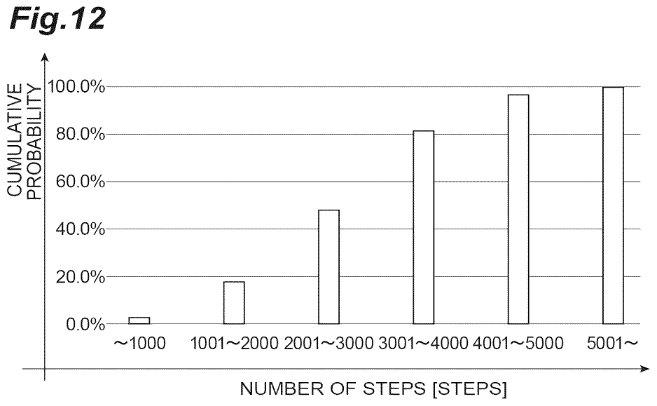

FIG. 10 is a diagram showing an example of a graph of the frequency of the number of steps. FIG. 11 is a diagram showing an example of the probability density distribution of the number of steps. FIG. 12 is a diagram showing an example of the cumulative distribution of the number of steps.

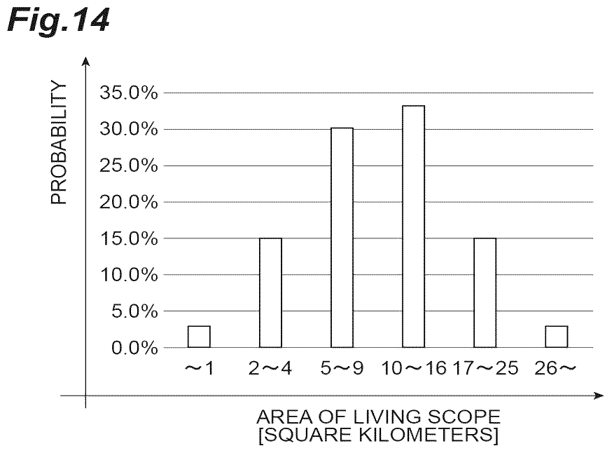

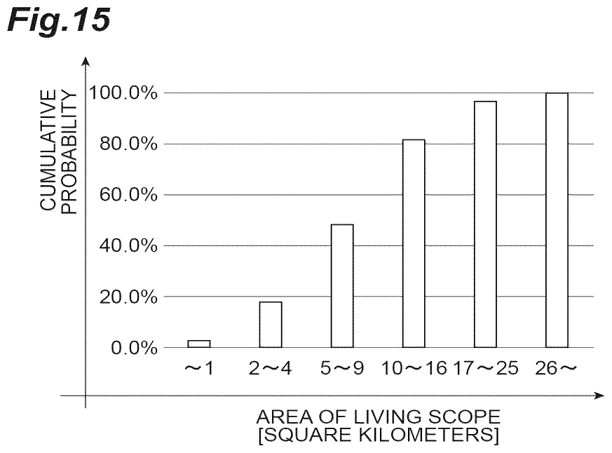

FIG. 13 is a diagram showing an example of a graph of the frequency of the area of the living scope. FIG. 14 is a diagram showing an example of the probability density distribution of the area of the living scope. FIG. 15 is a diagram showing an example of the cumulative distribution of the area of the living scope.

Next, a relationship between the overall trend and the cumulative distribution will be described.

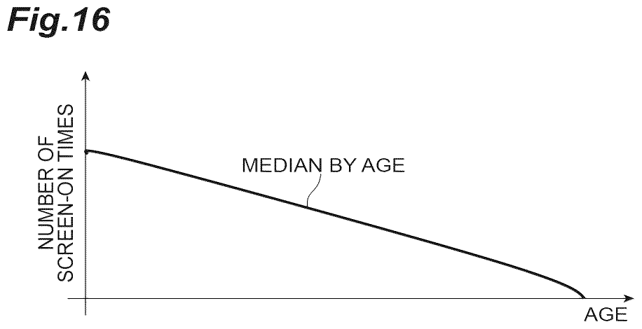

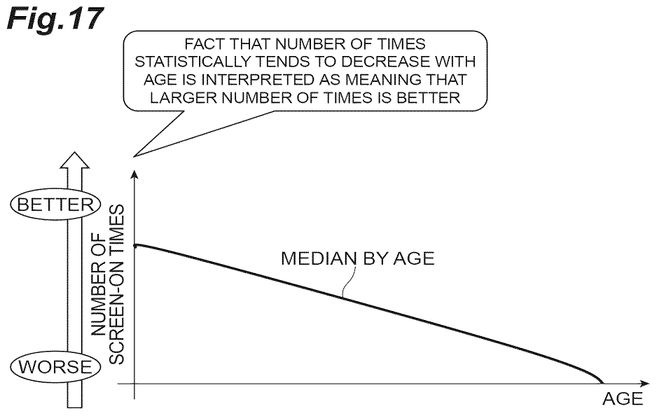

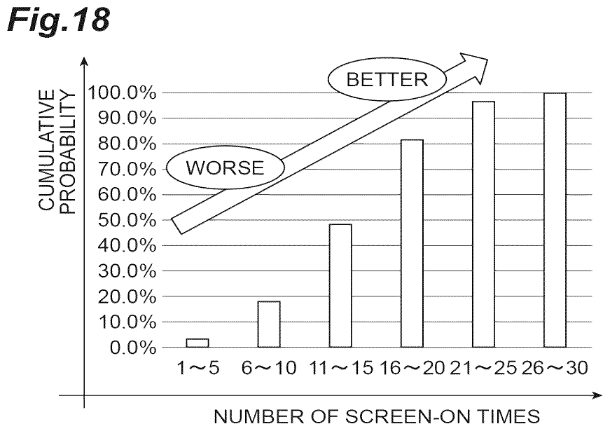

When there is a negative correlation with aging as an overall trend, the creation unit 12 creates a scoring model so that the larger the value (frequency), the larger the youthfulness degree (younger usage). FIG. 16 is a diagram showing an example of a graph of a median of the number of screen-on times by age. As shown in FIG. 16, the number of screen-on times, which is a log frequency, has a negative correlation with aging as an overall trend. In the youthfulness degree output device 1, the fact that the number of times statistically tends to decrease with age is interpreted as meaning that a larger number of times is better. FIG. 17 is a diagram showing an example of interpretation of the graph shown in FIG. 16. As shown in FIG. 17, a smaller number of screen-on times on the vertical axis of the graph is interpreted as being worse, and a larger number of screen-on times is interpreted as being better. FIG. 18 is a diagram showing an example of interpretation of the cumulative distribution of the number of screen-on times. As shown in FIG. 18, a smaller number of screen-on times is interpreted as being worse, and a larger number of screen-on times is interpreted as being better. In this case, the creation unit 12 creates a scoring model based on the following equation, for example.

$$given i \qquad [ Equation 2 ]$$

$$s = {\sum }_{x \leq i} P ( x )$$

In the above equation, the score s indicates a probability that the value (frequency) is equal to or less than i.

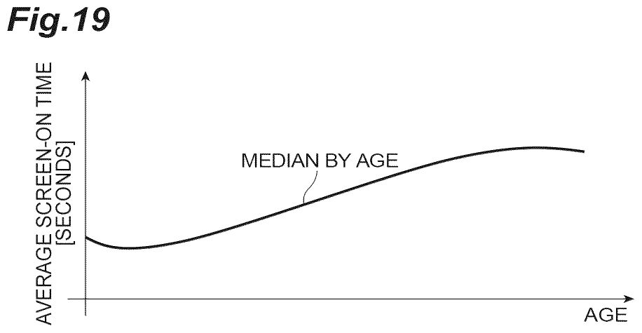

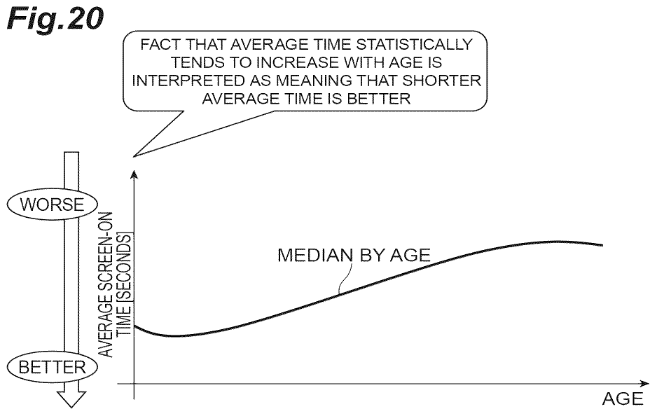

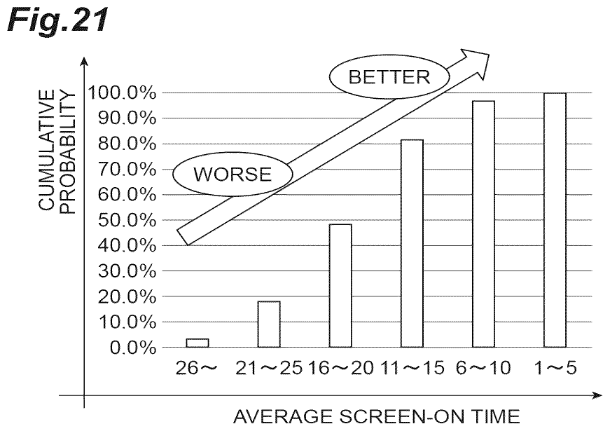

When there is a positive correlation with aging as an overall trend, the creation unit 12 creates a scoring model so that the smaller the value (frequency), the larger the youthfulness degree (younger usage). FIG. 19 is a diagram showing an example of a graph of a median of the average screen-on time by age. As shown in FIG. 19, the average screen-on time, which is a log frequency, has a positive correlation with aging as an overall trend. In the youthfulness degree output device 1, the fact that the average time statistically tends to increase with age is interpreted as meaning that a shorter average time is better. FIG. 20 is a diagram showing an example of interpretation of the graph shown in FIG. 19. As shown in FIG. 20, a larger average screen-on time on the vertical axis of the graph is interpreted as being worse, and a smaller average screen-on time is interpreted as being better. FIG. 21 is a diagram showing an example of interpretation of the cumulative distribution of the average screen-on time. In FIG. 21, the horizontal axis is reversed, and the stacking direction is reversed. As shown in FIG. 21, a larger average screen-on time is interpreted as being worse, and a smaller average screen-on time is interpreted as being better. In this case, the creation unit 12 creates a scoring model based on the following equation, for example.

$$given i \qquad [ Equation 3 ]$$

$$s = 1 - {\sum }_{x \leq i} P ( x )$$

In the above equation, the score s indicates a probability that the value (frequency) is equal to or greater than i.

As described above, when creating the scoring model by the creation unit 12, instead of simply using the cumulative distribution, different equations are used based on the relationship between the log frequency (data) and age.

An example of interpretation of the score s calculated from the cumulative distribution will be described. As described above, as an example of the interpretation of the score s calculated based on the correlation between age and log frequency, it can be interpreted as being at the top “(1-s) %” (also known as percentile score) compared to users of the same age (or the same generation). As another example of interpretation, it can be interpreted as “(s*100) points” in terms of a maximum score of 100 points.

Next, a process of creating a scoring model will be described.

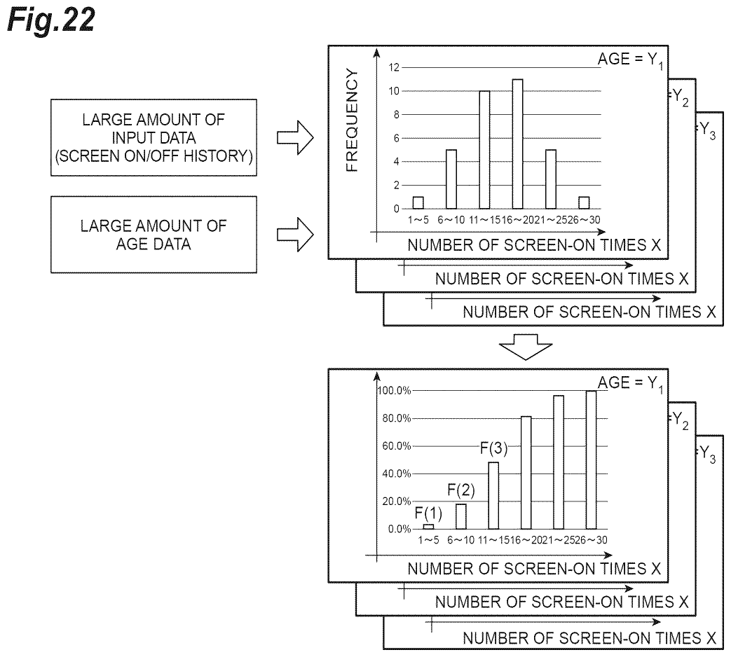

FIG. 22 is a diagram showing an example of the flow of a scoring model creation process. First, the acquisition unit 11 acquires a large amount of input data (log frequency, screen on/off history, and the like) and age data (the age of a user for whom the input data is intended). Then, the creation unit 12 counts a log frequency for each age based on the acquired large amount of input data and age data to create a probability density distribution P(X) (not shown) for each age. In addition, in the process of creating P(X), both X and Y may be converted into frequencies (An example of X: the number of screen-on times of 1 to 5 is defined as “1” and the number of screen-on times of 6 to 10 is defined as “2”. An example of Y: the 60s are defined as “6” and the 70s are defined as “7”). Then, the creation unit 12 creates a cumulative distribution F(X) for each age. Then, the creation unit 12 compiles the entire cumulative distribution for each age into a scoring model F(X, Y). During the compiling, the creation unit 12 defines a model when a predetermined age Y1 is given as F(X|Y₁), sets an integrated model F(X, Y)={F(X|Y₁), F(X|Y₂), F(X|Y₃), . . . }, and selects a model F(X|Yᵢ) for F(X, Y) according to the input age Y. That is, there are some precondition models F(X|Yᵢ) for which Yᵢ is given, and the sum of these is F(X, Y). This creation process flow is applied to each piece of input data.

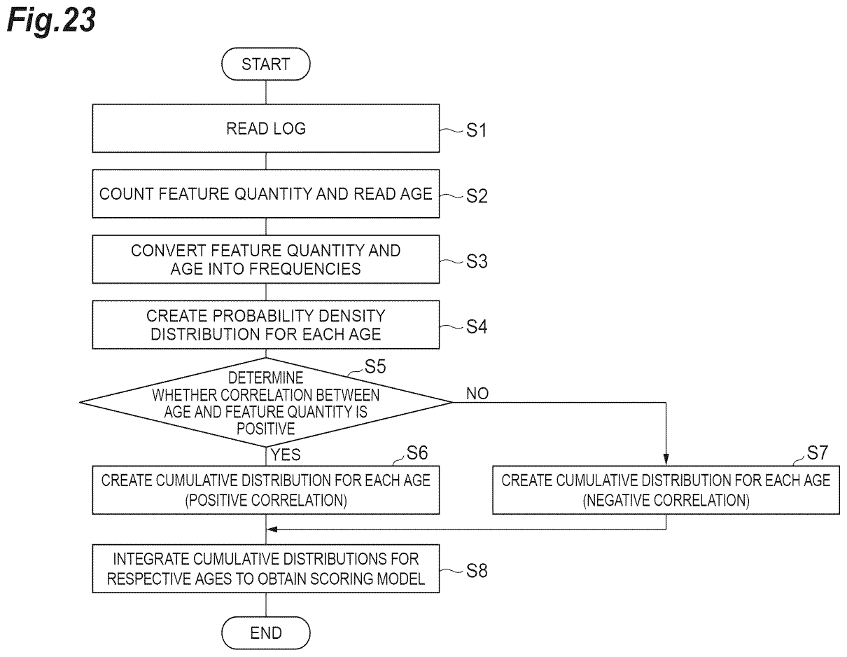

FIG. 23 is a flowchart showing an example of a scoring model creation process performed by the youthfulness degree output device 1. First, the acquisition unit 11 reads the log (step S1). Then, the creation unit 12 counts a feature quantity (log or log frequency) to read the age (step S2). Then, the creation unit 12 converts the feature quantity and the age into frequencies (step S3). Then, the creation unit 12 creates a probability density distribution for each age (step S4). Then, the creation unit 12 determines whether the correlation between the age and the feature quantity (log frequency) is positive or negative (step S5).

In addition, as a method for determining whether the correlation is positive or negative, for example, a Pearson's correlation coefficient may be used. For example, the correlation coefficient is determined as r=(covariance of variable X (log frequency) and variable Y (age))/standard deviation of variable X*standard deviation of variable Y).

When it is determined that the correlation is positive in S5 (S5: YES), the creation unit 12 creates a cumulative distribution (positive correlation) for each age (step S6). On the other hand, when it is determined that the correlation is negative in S5 (S5: NO), the creation unit 12 creates a cumulative distribution (negative correlation) for each age (step S7). Subsequent to S6 or S7, the creation unit 12 integrates the cumulative distributions for respective ages to obtain a scoring model (step S8). This scoring model creation process is applied to all log types to construct scoring models for all log types.

To add, when the correlation coefficient is positive, Y increases as X increases, so that it is better for the age Y to be smaller in the scoring model. Therefore, the creation unit 12 creates a cumulative distribution from the upper limit to the lower limit of the feature quantity so that a higher score is given as X decreases. On the other hand, when the correlation coefficient is negative, Y decreases as X increases. Therefore, the creation unit 12 creates a cumulative distribution from the lower limit to the upper limit of the feature quantity so that a higher score is given as X increases.

The output unit 13 outputs the youthfulness degree of the target user that is predicted by inputting the target user's age and log frequency acquired by the acquisition unit 11 (or stored in the storage unit 10) to the scoring model stored in the storage unit 10. The output from the output unit 13 may be displayed on a display, which is an output device 1006 (described later), or may be transmitted to another device through a communication device 1004 (described later). The output unit 13 may cause the storage unit 10 to store the youthfulness degree.

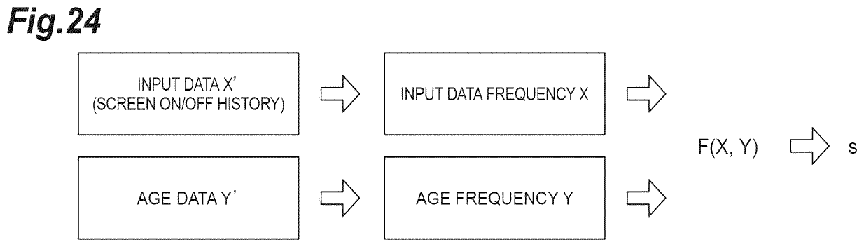

FIG. 24 is a diagram showing an example of the flow of a score calculation process. First, the acquisition unit 11 acquires input data X′ (log frequency, screen on/off history, and the like) and age data Y′ (the age of a user who is the target of the input data X′). Then, the output unit 13 or the youthfulness degree output device 1 converts the input data X′ into a frequency to obtain input data X, and converts the age data Y′ into a frequency to obtain age frequency Y. Then, the output unit 13 inputs the input data X and the age frequency Y to the scoring model F(X, Y) and outputs the score s. This calculation process is applied to each piece of input data.

The output unit 13 may output the youthfulness degree of the target user for each type that is predicted by inputting the target user's age and each of a plurality of types of log frequencies acquired by the acquisition unit 11 to a scoring model corresponding to the same type of log frequency stored in the storage unit 10. The output unit 13 may output a degree obtained by integrating the youthfulness degree of the target user for each type. The output unit 13 may output a degree obtained by integrating, for each similar type, the youthfulness degree of the target user for each type.

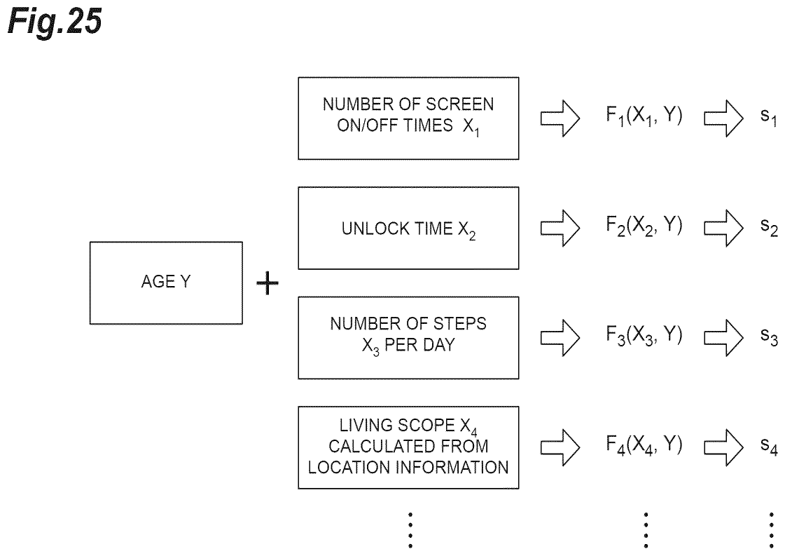

FIG. 25 is a diagram showing another example of the flow of the score calculation process. First, the acquisition unit 11 acquires age Y, the number of screen on/off times X₁, unlock time X₂, the number of steps taken per day X₃, living scope X₄ calculated from location information, . . . . X₁, X₂, X₃, X₄, . . . may be input after being converted into frequencies within the youthfulness degree output device 1 or the mobile terminal 2. Then, the output unit 13 applies X₁, X₂, X₃, X₄, . . . to the scoring model Fᵢ to calculate and output a score sᵢ for each type of log frequency. Specifically, these are output as s₁=F₁(X₁, Y), s₂=F₂(X₂, Y), s₃=F₃(X₃, Y), s₄=F₄ (X₄, Y), . . . . In addition, s₁, s₂, s₃, s₄, . . . may be cognitive function scores indicating a cognitive function. The output unit 13 may calculate and output a total score (a total youthfulness degree) by integrating s₁, s₂, s₃, s₄, . . . . The total score is expressed as, for example, s=average (s₁, s₂, . . . ) (average is an average value). In addition, the output unit 13 may count and output (visualize) each score for each item (similar type). For example, the score of an item related to the user's operation may be sₒₚₑᵣₐₜᵢₒₙ=average (s₁, s₂), and the score of an item related to the user's activity may be s_{activity}=average (s₃, s₄).

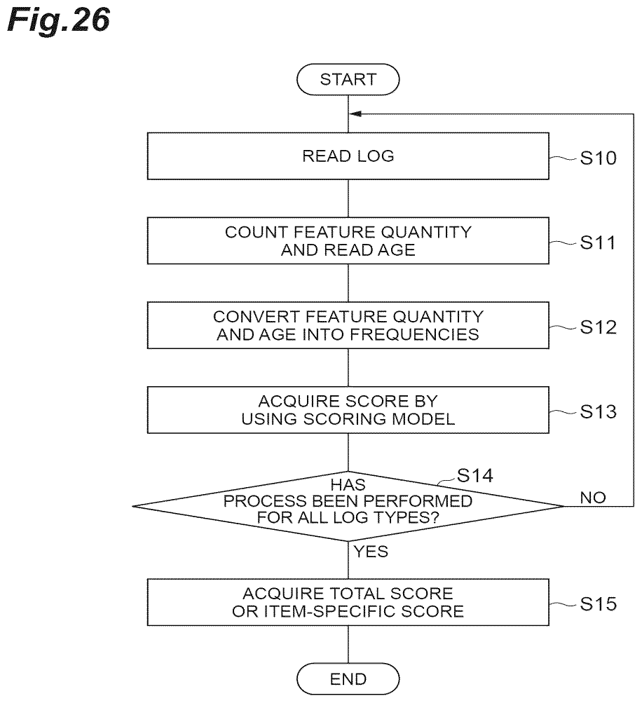

FIG. 26 is a flowchart showing an example of the score calculation process performed by the youthfulness degree output device 1. First, the acquisition unit 11 reads the log of the target user (step S10). Then, the output unit 13 counts the feature quantity (log or log frequency) of the target user to read the target user's age (step S11). Then, the output unit 13 converts the feature quantity and age of the target user into frequencies (step S12). Then, the output unit 13 inputs the feature quantity and age converted into frequencies in S12 to the scoring model to acquire and output a score (youthfulness degree) (step S13). Then, the output unit 13 determines whether or not the process has been performed for all log types (step S14). When it is determined that the process has not been performed for all log types in S14 (S14: NO), the process returns to S10. On the other hand, when it is determined that the process has been performed for all log types in S14 (S14: YES), the output unit 13 acquires and outputs a total score or an item-specific score (step S15).

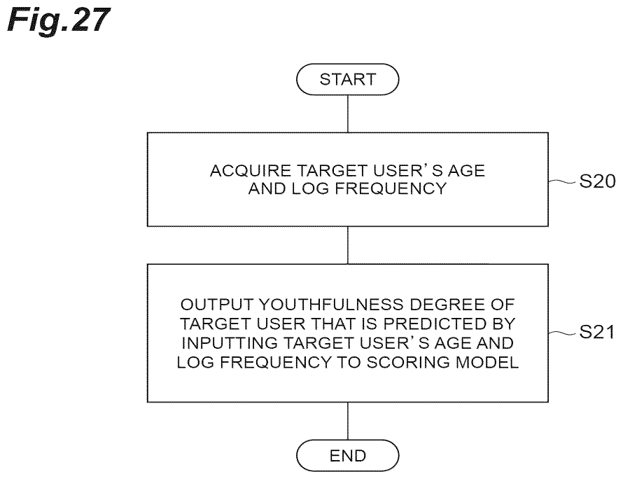

Next, an example of a youthfulness degree output process (youthfulness degree output method) performed by the youthfulness degree output device 1 will be described with reference to FIG. 27. FIG. 27 is a flowchart showing an example of the youthfulness degree output process performed by the youthfulness degree output device 1. First, the acquisition unit 11 acquires the target user's age and log frequency (step S20, acquisition step). Then, the output unit 13 outputs the youthfulness degree (score) of the target user that is predicted by inputting the target user's age and log frequency acquired in S20 to the scoring model stored in the storage unit 10 (step S21, output step).

Next, the function and effect of the youthfulness degree output device 1 according to the embodiment will be described.

The youthfulness degree output device 1 includes: the storage unit 10 that stores a scoring model (prediction model) that predicts the youthfulness degree of a user by inputting the user's age and a log frequency related to logs associated with the user's behavior obtained by the mobile terminal 2 (terminal) carried by the user; the acquisition unit 11 that acquires a target user's age and the log frequency; and the output unit 13 that outputs a youthfulness degree of the target user predicted by inputting the target user's age and the log frequency acquired by the acquisition unit 11 to the scoring model stored in the storage unit 10. The scoring model predicts a youthfulness degree based on a cumulative distribution function for each age calculated by counting the log frequency for each of a plurality of users by age. According to this configuration, the youthfulness degree of the target user that is predicted by inputting the target user's age and the log frequency to the prediction model is output. That is, it is possible to output the youthfulness degree of the user.

In addition, in the youthfulness degree output device 1, the scoring model may predict a youthfulness degree by applying the input log frequency to the cumulative distribution function of an input age among the cumulative distribution functions by age. According to this configuration, since a cumulative distribution function according to the target user's age is used, it is possible to output a more accurate youthfulness degree.

In addition, in the youthfulness degree output device 1, the cumulative distribution function may be calculated based on a correlation between age and the log frequency. According to this configuration, it is possible to output a more accurate youthfulness degree based on the correlation between age and the log frequency.

In addition, in the youthfulness degree output device 1, when there is a negative correlation between age and the log frequency, the cumulative distribution function may be calculated by accumulating a probability for each log frequency from a lower limit to an upper limit of the log frequency, and when there is a positive correlation between age and the log frequency, the cumulative distribution function may be calculated by accumulating a probability for each log frequency from an upper limit to a lower limit of the log frequency. According to this configuration, since a more accurate cumulative distribution function based on the negative or positive correlation between age and the log frequency is used, it is possible to output a more accurate youthfulness degree.

In addition, in the youthfulness degree output device 1, when there is a negative correlation between age and the log frequency, the scoring model may predict, as a youthfulness degree, a value based on a probability that the log frequency is equal to or lower than the input log frequency, and when there is a positive correlation between age and the log frequency, the scoring model may predict, as a youthfulness degree, a value based on a probability that the log frequency is equal to or higher than the input log frequency. According to this configuration, since a more accurate value based on the negative correlation or positive correlation between age and the log frequency is predicted as a youthfulness degree, it is possible to output a more accurate youthfulness degree.

In addition, the youthfulness degree output device 1 may further include the creation unit 12 that creates the scoring model, and the storage unit 10 may store the scoring model created by the creation unit 12. According to this configuration, it is possible to predict the youthfulness degree by using the created scoring model.

In addition, in the youthfulness degree output device 1, the storage unit 10 may stores the scoring model corresponding to each of a plurality of types of the log frequencies, the acquisition unit 11 may acquire the target user's age and the plurality of types of log frequencies, and the output unit 13 may output a youthfulness degree of the target user for each type predicted by inputting the target user's age and each of the plurality of types of log frequencies acquired by the acquisition unit 11 to the scoring model corresponding to the same type of log frequency stored in the storage unit 10. According to this configuration, it is possible to output a more accurate youthfulness degree based on a plurality of types of log frequencies.

In addition, in the youthfulness degree output device 1, the output unit 13 may output a degree obtained by integrating the youthfulness degree of the target user for each of the types. According to this configuration, it is possible to easily grasp the youthfulness degree.

In addition, in the youthfulness degree output device 1, the output unit 13 may output a degree obtained by integrating, for each similar type, the youthfulness degree of the target user for each of the types. According to this configuration, it is possible to grasp the youthfulness degree for each similar type.

According to the youthfulness degree output device 1, it is possible to output a youthfulness degree (perform scoring) by using a plurality of automatically obtainable logs (or log frequencies). In addition, in the youthfulness degree output device 1, it is possible to more accurately predict the youthfulness degree (improve the accuracy of scoring) by using age as an input parameter.

The youthfulness degree output device 1 of the present disclosure may have the following configuration.

[1] A youthfulness degree output device, including:
a storage unit that stores a prediction model for predicting a youthfulness degree of a user by inputting the user's age and a log frequency related to logs associated with the user's behavior obtained by a terminal carried by the user;
an acquisition unit that acquires a target user's age and the log frequency; and
an output unit that outputs a youthfulness degree of the target user predicted by inputting the target user's age and the log frequency acquired by the acquisition unit to the prediction model stored in the storage unit,
wherein the prediction model predicts a youthfulness degree based on a cumulative distribution function for each age calculated by counting the log frequency for each of a plurality of users by age.

[2] The youthfulness degree output device according to [1],
wherein the prediction model predicts a youthfulness degree by applying the input log frequency to the cumulative distribution function of an input age among the cumulative distribution functions by age.

[3] The youthfulness degree output device according to [1] or [2],
wherein the cumulative distribution function is calculated based on a correlation between age and the log frequency.

[4] The youthfulness degree output device according to any one of [1] to [3],
wherein, when there is a negative correlation between age and the log frequency, the cumulative distribution function is calculated by accumulating a probability for each log frequency from a lower limit to an upper limit of the log frequency, and
when there is a positive correlation between age and the log frequency, the cumulative distribution function is calculated by accumulating a probability for each log frequency from an upper limit to a lower limit of the log frequency.

[5] The youthfulness degree output device according to any one of [1] to [4],
wherein, when there is a negative correlation between age and the log frequency, the prediction model predicts, as a youthfulness degree, a value based on a probability that the log frequency is equal to or lower than the input log frequency, and
when there is a positive correlation between age and the log frequency, the prediction model predicts, as a youthfulness degree, a value based on a probability that the log frequency is equal to or higher than the input log frequency.

[6] The youthfulness degree output device according to any one of [1] to [5], further including:
a creation unit that creates the prediction model,
wherein the storage unit stores the prediction model created by the creation unit.

[7] The youthfulness degree output device according to any one of [1] to [6],
wherein the storage unit stores the prediction model corresponding to each of a plurality of types of the log frequencies,
the acquisition unit acquires the target user's age and the plurality of types of log frequencies, and
the output unit outputs a youthfulness degree of the target user for each type predicted by inputting the target user's age and each of the plurality of types of log frequencies acquired by the acquisition unit to the prediction model corresponding to the same type of log frequency stored in the storage unit.

[8] The youthfulness degree output device according to [7],
wherein the output unit outputs a degree obtained by integrating the youthfulness degree of the target user for each of the types.

[9] The youthfulness degree output device according to [7] or [8],
wherein the output unit outputs a degree obtained by integrating, for each similar type, the youthfulness degree of the target user for each of the types.

A youthfulness degree output method executed by a youthfulness degree output device including a storage unit that stores a prediction model for predicting a youthfulness degree of a user by inputting the user's age and a log frequency related to logs associated with the user's behavior obtained by a terminal carried by the user, the youthfulness degree output method including:
an acquisition step for acquiring a target user's age and the log frequency; and
an output step for outputting a youthfulness degree of the target user predicted by inputting the target user's age and the log frequency acquired in the acquisition step to the prediction model stored in the storage unit,
wherein the prediction model predicts a youthfulness degree based on a cumulative distribution function for each age calculated by counting the log frequency for each of a plurality of users by age.

[11] The youthfulness degree output method according to [10],
wherein the prediction model predicts a youthfulness degree by applying the input log frequency to the cumulative distribution function of an input age among the cumulative distribution functions by age.

[12] The youthfulness degree output method according to or [11],
wherein the cumulative distribution function is calculated based on a correlation between age and the log frequency.

[13] The youthfulness degree output method according to any one of [10] to [12],
wherein, when there is a negative correlation between age and the log frequency, the cumulative distribution function is calculated by accumulating a probability for each log frequency from a lower limit to an upper limit of the log frequency, and
when there is a positive correlation between age and the log frequency, the cumulative distribution function is calculated by accumulating a probability for each log frequency from an upper limit to a lower limit of the log frequency.

[14] The youthfulness degree output method according to any one of [10] to [13],
wherein, when there is a negative correlation between age and the log frequency, the prediction model predicts, as a youthfulness degree, a value based on a probability that the log frequency is equal to or lower than the input log frequency, and
when there is a positive correlation between age and the log frequency, the prediction model predicts, as a youthfulness degree, a value based on a probability that the log frequency is equal to or higher than the input log frequency.

[15] The youthfulness degree output method according to any one of [10] to [14], further including:
a creation step for creating the prediction model,
wherein the storage unit stores the prediction model created in the creation step.

[16] The youthfulness degree output method according to any one of [10] to [15],
wherein the storage unit stores the prediction model corresponding to each of a plurality of types of the log frequencies,
in the acquisition step, the target user's age and the plurality of types of log frequencies are acquired, and
in the output step, a youthfulness degree of the target user for each type predicted by inputting the target user's age and each of the plurality of types of log frequencies acquired in the acquisition step to the prediction model corresponding to the same type of log frequency stored in the storage unit is output.

[17] The youthfulness degree output method according to [16],
wherein, in the output step, a degree obtained by integrating the youthfulness degree of the target user for each of the types is output.

[18] The youthfulness degree output method according to [16] or [17],
wherein, in the output step, a degree obtained by integrating, for each similar type, the youthfulness degree of the target user for each of the types is output.

In addition, the block diagrams used in the description of the above embodiment show blocks in functional units. These functional blocks (configuration units) are realized by any combination of at least one of hardware and software. In addition, a method of realizing each functional block is not particularly limited. That is, each functional block may be realized using one physically or logically coupled device, or may be realized by connecting two or more physically or logically separated devices directly or indirectly (for example, using a wired or wireless connection) and using the plurality of devices. Each functional block may be realized by combining the above-described one device or the above-described plurality of devices with software.

Functions include determining, judging, calculating, computing, processing, deriving, investigating, searching, ascertaining, receiving, transmitting, outputting, accessing, resolving, selecting, choosing, establishing, comparing, assuming, expecting, regarding, broadcasting, notifying, communicating, forwarding, configuring, reconfiguring, allocating, mapping, assigning, and the like, but are not limited thereto.

For example, a functional block (configuration unit) that makes the transmission work is called a transmitting unit or a transmitter. In any case, as described above, the implementation method is not particularly limited.

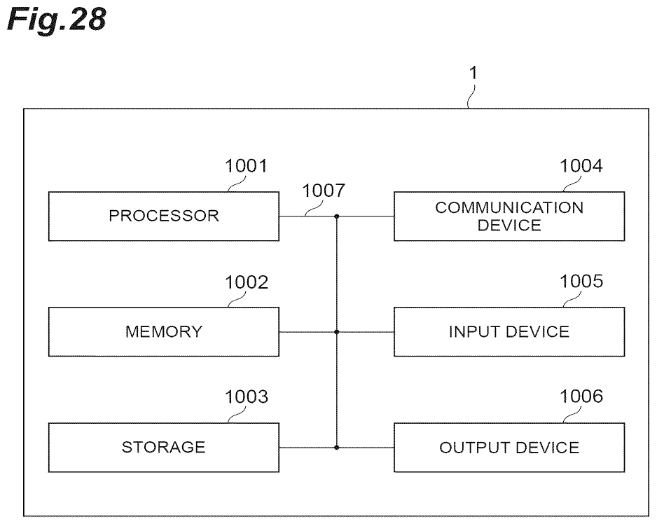

For example, the youthfulness degree output device 1 and the like according to an embodiment of the present disclosure may function as a computer that performs processing of the youthfulness degree output method of the present disclosure. FIG. 28 is a diagram showing an example of the hardware configuration of the youthfulness degree output device 1 according to an embodiment of the present disclosure. The youthfulness degree output device 1 described above may be physically configured as a computer device including a processor 1001, a memory 1002, a storage 1003, a communication device 1004, an input device 1005, an output device 1006, a bus 1007, and the like.

In addition, in the following description, the term “device” can be read as a circuit, a unit, and the like. The hardware configuration of the youthfulness degree output device 1 may include one or more devices for each device shown in the diagram, or may not include some devices.

Each function in the youthfulness degree output device 1 is realized by reading predetermined software (program) onto hardware, such as the processor 1001 and the memory 1002, so that the processor 1001 performs an operation and controlling communication by the communication device 1004 or controlling at least one of reading and writing of data in the memory 1002 and the storage 1003.

The processor 1001 controls the entire computer by operating an operating system, for example. The processor 1001 may be configured by a central processing unit (CPU) including an interface with a peripheral device, a control device, an operation device, a register, and the like. For example, the above-described acquisition unit 11, creation unit 12, and output unit 13 may be realized by the processor 1001.

In addition, the processor 1001 reads a program (program code), a software module, data, and the like into the memory 1002 from at least one of the storage 1003 and the communication device 1004, and executes various kinds of processing according to these. As the program, a program causing a computer to execute at least a part of the operation described in the above embodiment is used. For example, the acquisition unit 11, the creation unit 12, and the output unit 13 may be realized by a control program stored in the memory 1002 and operating in the processor 1001, or may be realized similarly for other functional blocks. Although it has been described that the various kinds of processes described above are performed by one processor 1001, the various kinds of processes described above may be performed simultaneously or sequentially by two or more processors 1001. The processor 1001 may be implemented by one or more chips. In addition, the program may be transmitted from a network through a telecommunication line.

The memory 1002 is a computer-readable recording medium, and may be configured by at least one of, for example, a ROM (Read Only Memory), an EPROM (Erasable Programmable ROM), an EEPROM (Electrically Erasable Programmable ROM), and a RAM (Random Access Memory). The memory 1002 may be called a register, a cache, a main memory (main storage device), and the like. The memory 1002 can store a program (program code), a software module, and the like that can be executed to implement the wireless communication method according to an embodiment of the present disclosure.

The storage 1003 is a computer-readable recording medium, and may be configured by at least one of, for example, an optical disk such as a CD-ROM (Compact Disc ROM), a hard disk drive, a flexible disk, and a magneto-optical disk (for example, a compact disk, a digital versatile disk, and a Blu-ray (Registered trademark) disk), a smart card, a flash memory (for example, a card, a stick, a key drive), a floppy (registered trademark) disk, and a magnetic strip. The storage 1003 may be called an auxiliary storage device. The storage medium described above may be, for example, a database including at least one of the memory 1002 and the storage 1003, a server, or other appropriate media.

The communication device 1004 is hardware (transmitting and receiving device) for performing communication between computers through at least one of a wired network and a wireless network, and is also referred to as, for example, a network device, a network controller, a network card, and a communication module. The communication device 1004 may be configured to include, for example, a high-frequency switch, a duplexer, a filter, a frequency synthesizer, and the like in order to realize at least one of frequency division duplex (FDD) and time division duplex (TDD), for example. For example, the above-described acquisition unit 11, creation unit 12, and output unit 13 may be realized by the communication device 1004.

The input device 1005 is an input device (for example, a keyboard, a mouse, a microphone, a switch, a button, and a sensor) for receiving an input from the outside. The output device 1006 is an output device (for example, a display, a speaker, and an LED lamp) that performs output to the outside. In addition, the input device 1005 and the output device 1006 may be integrated (for example, a touch panel).

In addition, respective devices, such as the processor 1001 and the storage device 1002, are connected to each other by the bus 1007 for communicating information. The bus 1007 may be configured using a single bus, or may be configured using a different bus for each device.

In addition, the youthfulness degree output device 1 may be configured to include hardware, such as a microprocessor, a digital signal processor (DSP), an ASIC (Application Specific Integrated Circuit), a PLD (Programmable Logic Device), and an FPGA (Field Programmable Gate Array), and some or all of the functional blocks may be realized by the hardware. For example, the processor 1001 may be implemented by using at least one of these hardware components.

The notification of information is not limited to the aspects/embodiments described in the present disclosure, and may be performed using other methods.

Each aspect/embodiment described in the present disclosure may be applied to at least one of systems, which use LTE (Long Term Evolution), LTE-A (LTE-Advanced), SUPER 3G, IMT-Advanced, 4G (4th generation mobile communication system), 5G (5th generation mobile communication system), FRA (Future Radio Access), NR (new Radio), W-CDMA (registered trademark), GSM (registered trademark), CDMA2000, UMB (Ultra Mobile Broadband), IEEE 802.11 (Wi-Fi (registered trademark)), IEEE 802.16 (WiMAX (registered trademark)), IEEE 802.20, UWB (Ultra-WideBand), Bluetooth (registered trademark), and other appropriate systems, and next-generation systems extended based on these. In addition, a plurality of systems may be combined (for example, a combination of 5G and at least one of LTE and LTE-A) to be applied.

In the processing procedure, sequence, flowchart, and the like in each aspect/embodiment described in the present disclosure, the order may be changed as long as there is no contradiction. For example, for the methods described in the present disclosure, elements of various steps are presented using an exemplary order. However, the present invention is not limited to the specific order presented.

Information or the like that is input and output may be stored in a specific place (for example, a memory) or may be managed using a management table. The information or the like that is input and output can be overwritten, updated, or added. The information or the like that is output may be deleted. The information or the like that is input may be transmitted to another device.

The judging may be performed based on a value (0 or 1) expressed by 1 bit, may be performed based on the Boolean value (Boolean: true or false), or may be performed by numerical value comparison (for example, comparison with a predetermined value).

Each aspect/embodiment described in the present disclosure may be used alone, may be used in combination, or may be switched and used according to execution. In addition, the notification of predetermined information (for example, notification of “X”) is not limited to being explicitly performed, and may be performed implicitly (for example, without the notification of the predetermined information).

While the present disclosure has been described in detail, it is apparent to those skilled in the art that the present disclosure is not limited to the embodiments described in the present disclosure. The present disclosure can be implemented as modified and changed aspects without departing from the spirit and scope of the present disclosure defined by the description of the claims. Therefore, the description of the present disclosure is intended for illustrative purposes, and has no restrictive meaning to the present disclosure.

Software, regardless of whether this is called software, firmware, middleware, microcode, a hardware description language, or any other name, should be interpreted broadly to mean instructions, instruction sets, codes, code segments, program codes, programs, subprograms, software modules, applications, software applications, software packages, routines, subroutines, objects, executable files, execution threads, procedures, functions, and the like.

In addition, software, instructions, information, and the like may be transmitted and received through a transmission medium. For example, in a case where software is transmitted from a website, a server, or other remote sources using at least one of the wired technology (coaxial cable, optical fiber cable, twisted pair, digital subscriber line (DSL), and the like) and the wireless technology (infrared, microwave, and the like), at least one of the wired technology and the wireless technology is included within the definition of the transmission medium.

The information, signals, and the like described in the present disclosure may be expressed using any of a variety of different technologies. For example, data, instructions, commands, information, signals, bits, symbols, and chips that can be referred to throughout the above description may be represented by voltage, current, electromagnetic waves, magnetic field or magnetic particles, light field or photon, or any combination thereof.

In addition, the terms described in the present disclosure and the terms necessary for understanding the present disclosure may be replaced with terms having the same or similar meaning.

The terms “system” and “network” used in the present disclosure are used interchangeably.

In addition, the information, parameters, and the like described in the present disclosure may be expressed using an absolute value, may be expressed using a relative value from a predetermined value, or may be expressed using another corresponding information.

The names used for the parameters described above are not limiting names in any way. In addition, equations and the like using these parameters may be different from those explicitly disclosed in the present disclosure.

The terms “determining” used in the present disclosure may involve a wide variety of operations. For example, “determining” can include considering judging, calculating, computing, processing, deriving, investigating, looking up (search, inquiry) (for example, looking up in a table, database, or another data structure), and ascertaining as “determining”. In addition, “determining” can include considering receiving (for example, receiving information), transmitting (for example, transmitting information), input, output, accessing (for example, accessing data in a memory) as “determining”. In addition, “determining” can include considering resolving, selecting, choosing, establishing, comparing, and the like as “determining”. In other words, “determining” can include considering any operation as “determining”. In addition, “determining” may be read as “assuming”, “expecting”, “considering”, and the like.

The terms “connected” and “coupled” or variations thereof mean any direct or indirect connection or coupling between two or more elements, and can include a case where one or more intermediate elements are present between two elements “connected” or “coupled” to each other. The coupling or connection between elements may be physical, logical, or a combination thereof. For example, “connection” may be read as “access”. When used in the present disclosure, two elements can be considered to be “connected” or “coupled” to each other using at least one of one or more wires, cables, and printed electrical connections and using some non-limiting and non-inclusive examples, such as electromagnetic energy having wavelengths in a radio frequency domain, a microwave domain, and a light (both visible and invisible) domain.

The description “based on” used in the present disclosure does not mean “based only on” unless otherwise specified. In other words, the description “based on” means both “based only on” and “based at least on”.

Any reference to elements using designations such as “first” and “second” used in the present disclosure does not generally limit the quantity or order of the elements. These designations can be used in the present disclosure as a convenient method for distinguishing between two or more elements. Therefore, references to first and second elements do not mean that only two elements can be adopted or that the first element should precede the second element in any way.

“Means” in the configuration of each device described above may be replaced with “unit”, “circuit”, “device”, and the like.

When “include”, “including”, and variations thereof are used in the present disclosure, these terms are intended to be inclusive similarly to the term “comprising”. In addition, the term “or” used in the present disclosure is intended not to be an exclusive-OR.

In the present disclosure, in a case where articles, for example, a, an, and the in English, are added by translation, the present disclosure may include that nouns subsequent to these articles are plural.

In the present disclosure, the expression “A and B are different” may mean “A and B are different from each other”. In addition, the expression may mean that “A and B each are different from C”. Terms such as “separate”, “coupled” may be interpreted similarly to “different”.

### REFERENCE SIGNS LIST

1 . . . youthfulness degree output device, 2 . . . mobile terminal, 3 . . . youthfulness degree output system, 10 . . . storage unit, 11 . . . acquisition unit, 12 . . . creation unit, 13 . . . output unit, 1001 . . . processor, 1002 . . . memory, 1003 . . . storage, 1004 . . . communication device, 1005 . . . input device, 1006 . . . output device, 1007 . . . bus.

1. A youthfulness degree output device, comprising processing circuitry configured to:
store a prediction model for predicting a youthfulness degree of a user by inputting the user's age and a log frequency related to logs associated with the user's behavior obtained by a terminal carried by the user;
acquire a target user's age and the log frequency; and
output a youthfulness degree of the target user predicted by inputting the acquired target user's age and the acquired log frequency to the stored prediction model,
wherein the prediction model predicts a youthfulness degree based on a cumulative distribution function for each age calculated by counting the log frequency for each of a plurality of users by age.

2. The youthfulness degree output device according to claim 1,
wherein the prediction model predicts a youthfulness degree by applying the input log frequency to the cumulative distribution function of an input age among the cumulative distribution functions by age.

3. The youthfulness degree output device according to claim 1,
wherein the cumulative distribution function is calculated based on a correlation between age and the log frequency.

4. The youthfulness degree output device according to claim 1,
wherein, when there is a negative correlation between age and the log frequency, the cumulative distribution function is calculated by accumulating a probability for each log frequency from a lower limit to an upper limit of the log frequency, and
when there is a positive correlation between age and the log frequency, the cumulative distribution function is calculated by accumulating a probability for each log frequency from an upper limit to a lower limit of the log frequency.

5. The youthfulness degree output device according to claim 1,
wherein, when there is a negative correlation between age and the log frequency, the prediction model predicts, as a youthfulness degree, a value based on a probability that the log frequency is equal to or lower than the input log frequency, and
when there is a positive correlation between age and the log frequency, the prediction model predicts, as a youthfulness degree, a value based on a probability that the log frequency is equal to or higher than the input log frequency.

6. The youthfulness degree output device according to claim 1, wherein the processing circuitry is further configured to:
create the prediction model,
wherein the processing circuitry is configured to store the created prediction model.

7. The youthfulness degree output device according to claim 1,
wherein the processing circuitry is configured to store the prediction model corresponding to each of a plurality of types of the log frequencies,
acquire the target user's age and the plurality of types of log frequencies, and
output a youthfulness degree of the target user for each type predicted by inputting the acquired target user's age and each of the acquired plurality of types of log frequencies to the stored prediction model corresponding to the same type of log frequency.

8. The youthfulness degree output device according to claim 7,
wherein the processing circuitry is configured to output a degree obtained by integrating the youthfulness degree of the target user for each of the types.

9. The youthfulness degree output device according to claim 7,
wherein the processing circuitry is configured to output a degree obtained by integrating, for each similar type, the youthfulness degree of the target user for each of the types.

10. A youthfulness degree output method executed by a youthfulness degree output device which stores a prediction model for predicting a youthfulness degree of a user by inputting the user's age and a log frequency related to logs associated with the user's behavior obtained by a terminal carried by the user, the youthfulness degree output method comprising:
an acquisition step for acquiring a target user's age and the log frequency; and
an output step for outputting a youthfulness degree of the target user predicted by inputting the target user's age and the log frequency acquired in the acquisition step to the stored prediction model,
wherein the prediction model predicts a youthfulness degree based on a cumulative distribution function for each age calculated by counting the log frequency for each of a plurality of users by age.
